(19) United States
(12) Patent Application Publication
(10) Pub. No.: US 2015/0328447 A1
(43) Pub. Date: Nov. 19, 2015

(54) Enhancement of Sensory Sensitivity by Transcranial Alternating Current Stimulation

(71) Applicant: Rutgers, The State University of New Jersey, New Brunswick, NJ (US)

(72) Inventors: Bart Krekelberg, New Brunswick, NJ (US); Kohitij Kar, New Brunswick, NJ (US)

(21) Appl. No.: 14/711,171

(22) Filed: May 13, 2015

Related U.S. Application Data
(60) Provisional application No. 61/992,301, filed on May 13, 2014.

(51) Int. Cl. A61N 1/04; A61N 1/36

(52) U.S. Cl. CPC A61N 1/0408; A61N 1/0484; A61N 1/36025

(57) ABSTRACT

A system for enhancing a sensory modality of a subject including a first scalp electrode configured to be placed above the sensory cortex of the subject and a second electrode configured to be placed elsewhere on the subject. A power source is configured to apply low levels of alternating current above 5 Hz between the first and second electrodes. A method of sensory enhancement is also provided.

This application claims the benefit of U.S. Provisional Application No. 61/992,301, filed on May 13, 2014, the contents of which are incorporated herein by reference.

### FIELD OF THE INVENTION

This invention relates to transcranial alternating current stimulation (tACS). More specifically, the invention relates to the use of tACS to enhance sensory sensitivity.

### BACKGROUND OF THE INVENTION

There is rapidly growing interest in using tACS to modulate brain activity in both clinical applications and cognitive neuroscience research. For instance, tACS has been claimed to suppress Parkinsonian tremors, entrain motor performance, aid recovery after stroke, and improve learning and memory, to name just a few. The mechanisms that underlie these long-term effects, however, remain poorly understood.

Even though applied fields clearly modulate membrane polarization, the long-term effects of electrical stimulation may not be the direct consequence of this polarization, but the indirect consequence of changes in plasticity induced by the stimulation.

### SUMMARY OF THE INVENTION

tACS is used in clinical applications and basic neuroscience research. Although its behavioral effects are evident from prior reports, current understanding of the mechanisms that underlie these effects is limited. The inventors used motion perception, a percept with relatively well-known properties and underlying neural mechanisms to investigate tACS mechanisms. More specifically, the inventors used visual motion discrimination in humans to investigate this view. This model system has the advantage that its neural mechanisms are relatively well understood, that a specific cortical area (hMT+) has been identified to play a critical role, and that a large arsenal of objective measures for behavioral report are available for its study.

Healthy human volunteers showed a surprising improvement in motion sensitivity when visual stimuli were paired with 10 Hz tACS. In addition, tACS reduced the motion-after effect, and this reduction was correlated with the improvement in motion sensitivity. Electrical stimulation had no consistent effect when applied before presenting a visual stimulus or during recovery from motion adaptation. Together, these findings suggest that perceptual effects of tACS result from an attenuation of adaptation. The techniques herein may be utilized for enhancement of visual detection as well as other sensory modalities. For instance, tACS could help to improve the detection of any faint visual stimulus, a faint sound, touch or smell.

Important consequences for the practical use of tACS follow from the inventors' work. First, because this mechanism interferes only with adaptation, this suggests that tACS can be targeted at subsets of neurons (by adapting them), even when the applied currents spread widely throughout the brain. Second, by interfering with adaptation, this mechanism provides a means by which electrical stimulation can generate behavioral effects that outlast the stimulation.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 illustrates exemplary experimental paradigms. The lightning bolt represents the application of tACS. In each paradigm, subjects indicated the perceived direction of motion of the ‘Test’ stimulus by pressing the up (↑) or down (↓) button.

FIG. 2 illustrates various conditions and results for motion discrimination tasks. In section A, the top portion illustrates the exemplary task design and the bottom portion illustrates the psychometric functions computed for an example subject with (thick black curve) and without (thin gray curve) tACS. Section B illustrates the change in sensitivity after application of tACS (for all eight subjects). Error bars indicate bootstrapped standard deviations of the sensitivity estimate. Sections C and D are similar to sections A and B but are for the ipsilateral motion discrimination task. In these illustrations, an * indicates a significant change in sensitivity for an individual subject. The data shows that tACS improved motion sensitivity in the contralateral, but not in the ipsilateral hemifield.

FIG. 3 illustrates various conditions and results for motion discrimination tasks. In section A, the top portion illustrates the exemplary task design for contralateral condition and the bottom portion illustrates psychometric functions computed for an example subject with (thick black curve) and without (thin gray curve) tACS. The dashed psychometric curve represents the performance in the unadapted condition. The horizontal error bars refer to the bootstrapped standard deviation of the PSE estimate. Section B illustrates the change in PSE after application of contralateral tACS (for all ten subjects). Error bars indicate bootstrapped standard deviations of the PSE estimate. Section C illustrates changes in PSE with tACS during adaptation (PSE_{adapt,tACS}-PSE_{adapt}) as a function of MAE induced by adaptation without tACS (PSE_{adapt}-PSE_{unadapt)}. The black solid line is a linear orthonormal fit to the data points. Sections D-F are similar to sections A-C but are for the ipsilateral condition. In these illustrations, an * indicates a significant change in PSE for an individual subject. Contralateral, but not ipsilateral, tACS reduced motion adaptation proportional to the amount of adaptation induced without tACS.

FIG. 4 illustrates sensitivity changes during motion adaptation. Section A illustrates changes in sensitivity with contralateral tACS during adaptation (Sensitivity_{adapt,tACS}-Sensitivity_{adapt}) as a function of sensitivity changes induced by adaptation without tACS (Sensitivity_{unadapt}-Sensitivity_{adapt}). Section B illustrates changes in sensitivity with ipsilateral tACS. The black solid lines are linear orthonormal fits to the data points. Sensitivity changes induced by adaptation were attenuated by contralateral tACS, but unaffected by ipsilateral tACS.

FIG. 5 illustrates reaction time (RT) changes during tACS. Section A illustrates changes in reaction time (ΔRT) in the speed detection task induced by tACS as a function of reaction times without tACS. The bold line is a robust locally weighted polynomial regression fit to the data. The vertical error bars represent the standard error. Section B is similar to section A, but for ipsilateral stimulation. tACS reduced reaction times, but only for contralateral visual stimuli.

FIG. 6 illustrates the experimental setup and procedure for macaque monkeys. Section a) illustrates the visual paradigm. On each trial a dot pattern (random or coherent motion) was presented for 3 s followed by a blank period of 300 ms, and then a 300 ms coherent dot pattern (moving in one of eight evenly spaced directions). Monkeys fixated a dot at the center of the monitor screen. Dot patterns were centered on the RF of the neuron being recorded. The two tACS electrodes were placed on either side of the recording chamber. Section b) illustrates the local field potentials recorded during an example session without tACS. Section c) illustrates the local field potentials recorded during the same session as b) with tACS. The LFPs in the latter condition were dominated by stimulation artifacts. Hence, the inventors only analyzed data obtained at least 150 ms after tACS offset (shading).

FIG. 7 illustrates the effects of tACS on direction tuning curves in four example neurons. Each panel shows tuning curve estimates of an example neuron in the four experimental conditions (black—unadapted; green—unadapted with tACS; blue—adapted; red—adapted with tACS). The open circles represent the mean firing rate across trials and the error bars indicate the standard error. The bold lines are tuning functions fitted to the mean firing rates per condition (see Methods). Section a) illustrates tACS attenuated the adaptation-induced suppression in tuning amplitude. Section b) illustrates tACS attenuated the adaptation-induced facilitation in tuning amplitude. Section c) illustrates tACS reduced the adaptation-induced broadening of the tuning curve. Section d) illustrates tACS reduced the adaptation-induced sharpening of the tuning curve. No consistent tACS-induced changes were observed in the unadapted condition (green curves). Thus, tACS consistently attenuated adaptation-induced changes in neuronal tuning properties.

FIG. 8 illustrates population analysis of tACS-induced changes in tuning properties. Section a) illustrates a comparison of the tuning amplitude change induced by tACS (during adaptation) with the tuning amplitude change induced by adaptation. Each dot represents a single neuron. Lines show the result of an orthogonal linear regression. Section b) is the same as Section a), but comparing changes in tuning width. Section c) illustrates a comparison of the tACS-induced change in tuning amplitude in the unadapted conditions with tACS-induced change in tuning amplitude in the adapted conditions. Section d) is the same as Section c), but comparing changes in tuning width. This figure shows that the tuning curve changes induced by adaptation (and only those changes) are partially undone when adaptation is combined with tACS. In other words, tACS consistently attenuated adaptation.

FIG. 9 illustrates that tACS-induced effects depended on the level of adaptation. Section a) illustrates the change in tuning amplitude (TA) after adaptation as a function of the difference between the adapter direction and the preferred direction of the neuron. The asterisk (*) indicates a significant difference (p<0.05) from 0. Section b) illustrates the effect of tACS on TA following adaptation. Section c) illustrates the change in tuning width (TW) after adaptation as a function of the difference between the adapter direction and the preferred direction of the neuron. Section d) illustrates the effect of tACS on TW following adaptation. Sections e) and f) show the numbers of neurons recorded in each of the groups. The smaller number of neurons adapted on the flank of their direction tuning curve is consistent with the recording strategy to choose an adapter close to the preferred direction of the cell under study (and thereby maximize the adaptation effect). Overall, this figure shows that the attenuation by tACS was large when the effect of adaptation was large.

FIG. 10 illustrates the influence of tACS on local field potentials. Section a) illustrates the LFP response evoked by the adapter (dashed, unadaptPRE) and the test stimuli (solid: unadapt (black), adapt (blue), unadapttACS (green), adapttACS (green)). Data were averaged over all sites (N=76). Shading shows standard errors. Adaptation reduced the N1 and N2 components (compare dashed black with solid black and blue curves). tACS attenuated the reduction of the N2 component, and also increased the magnitude of the evoked LFP after 100 ms (compare dashed black with red and green curves). Section b) illustrates the normalized power spectrum of the LFP (See Methods). LFP power was significantly reduced in the test phase (compare dashed black with solid blue and black curves), but tACS attenuated this reduction (red and green curve). There was no evidence for a frequency-specific (i.e. 10 Hz) entrainment of the LFP during the test stimuli.

FIG. 11 illustrates the broadband LFP power changes after adaptation and tACS. Section a) illustrates the change in LFP power after adaptation, as a function of the difference between the sites' preferred direction (gamma (30-120 Hz)) tuning and the adapters' direction of motion. Sites adapted near their preferred direction of motion show a greater decrease in broadband spectral power. Section b) illustrates the change in LFP power due to tACS applied during the adaptation phase. tACS increased power in sites that adapted most.

FIG. 12 is a perspective view illustrating a headgear in accordance with an exemplary embodiment of the invention.

### DETAILED DESCRIPTION OF THE INVENTION

Certain terminology is used herein for convenience only and is not to be taken as a limitation on the present invention. The following describes preferred embodiments of the present invention. However, it should be understood, based on this disclosure, that the invention is not limited by the preferred embodiments described herein.

The inventors first hypothesized that direct, tACS-induced perturbations should generate impairments in motion discrimination, because such perturbations are uninformative with respect to the direction of visual motion. The experiments rejected this hypothesis; instead the inventors found that subjects were better at motion direction discrimination during the application of tACS. Puzzled by this unexpected improvement in performance, the inventors hypothesized that tACS could have prevented the reduction in motion discrimination performance that has previously been reported to occur for prolonged stimulus presentations.

In a second set of experiments, the inventors tested this hypothesis using a standard motion adaptation paradigm. In such paradigms, a few seconds of exposure to, for instance, an upward moving pattern, generates the illusory percept of downward motion in a subsequent stationary or random motion stimulus. Adaptation typically reduces motion discrimination performance. The behavioral effects of motion adaptation have been linked to neural adaptation in the middle temporal area, and the time scale at which these effects persist (tens of seconds) suggest that they rely on plastic changes such as synaptic depression, or long-term after hyperpolarization. Consistent with the inventors' hypothesis, the experiments confirmed that tACS during the presentation of the visual motion stimulus (i.e., during the induction of adaptation) attenuated motion adaptation.

In a third set of experiments, the inventors investigated the influence of tACS in area MT of the macaque. The inventors recorded extracellular signals in area MT while applying tACS using scalp electrodes. The inventors investigated changes in individual neurons' firing rates as well as measures of synchronous population activity reflected in the local field potentials (LFP). To avoid misinterpreting electrical artifacts from stimulation as changes in neural activity, the analysis was restricted to the period following transcranial stimulation. The data shows that tACS attenuated the effects of neural adaptation. These effects included changes in the spiking response amplitude and direction tuning width, and changes in the amplitude and power spectrum of the LFP.

Taken together, the experiments suggest a novel mode of action of tACS; the attenuation of adaptation. In the discussion, the inventors address the implications of these findings for using tACS and speculate about the underlying neural mechanisms.

Second Experiments—Human Behavioral Analysis

Method—Electrode Placement—One electrode was placed above the canonical location of left hMT+; PO7-PO3 in the 10-20 system. The other electrode was placed on the vertex (Cz). In the main experiments, the parietal electrode was contralateral to the visual stimuli. In the ipsilateral control experiments, the electrode was placed above the hMT+ that was ipsilateral to the visual stimuli.

Subjects—Fifteen subjects participated in the experiments (eight female; fourteen naïve and one experimenter in total; 9 subjects for the motion discrimination task, 10 subjects for the motion adaptation task, 10 subjects for the recovery task and 8 subjects for the pre stimulus tACS task). They gave written consent and had normal or corrected to normal vision. This study was conducted according to the principles expressed in the Declaration of Helsinki and approved by the Institutional Review Board of Rutgers University.

Apparatus—tACS was delivered through a STG4002 stimulus generator. The stimulating electrodes were prepared as saline soaked sponges attached to conductive rubber electrodes (3″ diameter). The inventors used a sinusoidal current (1 mA peak to peak) at a frequency of 10 Hz. For safety reasons, the maximum voltage to produce the transcranial current was limited to 20V. The maximum current intensity was 0.5 mA and the electrode surface area was 45.6 cm². All eye movements were recorded using an eye tracker (Eyelink II V 2.2) at 500 Hz. Stimulus presentations and the triggering of stimulation were under the control of Neurostim (http://neurostim.sourceforge.net).

Visual Stimuli—Stimuli were presented on a CRT monitor (Sony FD Trinitron) with a resolution of 1024×768 pixels at a refresh rate of 120 Hz. The main motion stimulus was a dynamic random dot kinematogram (RDK) consisting of 700 dots with an infinite lifetime and an effective diameter of 1.5 pixels using spatial dithering (OpenGL point size of 1.5). The dots were restricted inside a circular aperture of radius 5° centered 7° to the left or right of the center of the screen. The luminance of the dots was 30 cd/m², the background 0.4 cd/m². The dots moved at a constant speed of 3°/sec. except during the speed change detection task (used to control for attention during adaptation; see below) when they moved at 6°/sec. for a brief (200 ms) period of time. The inventors refer to the percentage of dots moving in the same direction (positive coherence: up, negative coherence: down) as the coherence. The remainder of the dots moved in randomly chosen directions.

The RDK was used to construct the following five types of motion stimuli:

Long Adapter: RDK with dots moving upward with a coherence of 100% for 40 seconds.

Top-up Adapter: RDK with dots moving upward with a coherence of 100% for 4 seconds.

Test: RDK with different levels of coherence, presented for 1 second.

Long Test: RDK with different levels of coherence, presented for 4 seconds.

Random: RDK with all dots moving in a randomly chosen direction (0% coherence).

Experimental Procedures - Subjects were seated in a dark room at a distance of 57 cm from the center of the monitor. Head movements were restricted by a molded bite bar. The subjects indicated their response using the keyboard. Fixation of a central red dot was monitored and trials in which the eye strayed beyond a virtual window of 2° were discarded.

Because transcranial electrical stimulation has been shown to have long lived effects, experimental conditions with and without stimulation could not be interleaved. The minimal time to start blocks of trials without stimulation after tACS had been administered for any paradigm was 24 hours.

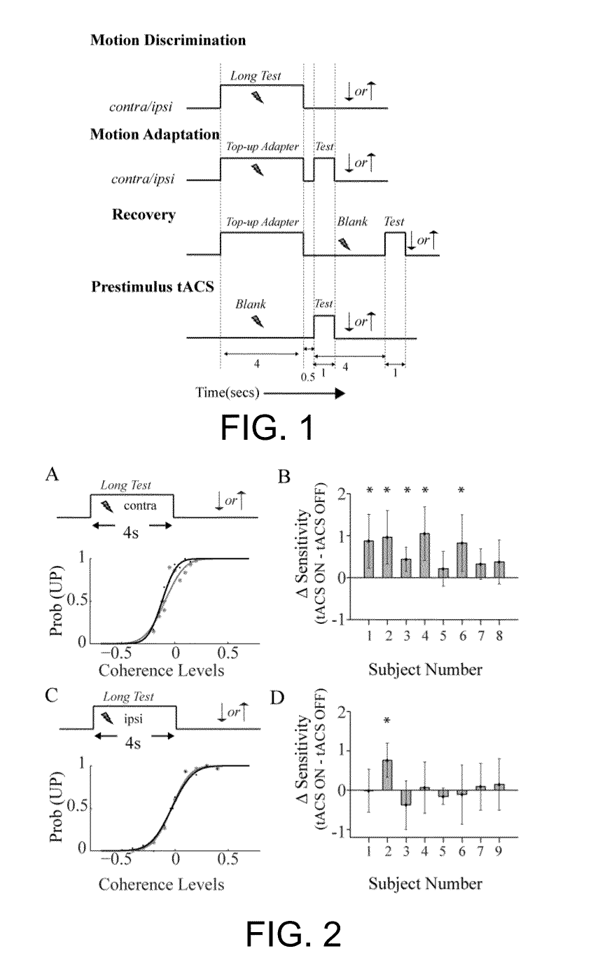

Behavioral Tasks—In each of the experiments, the subjects' task was to indicate the perceived global direction of motion of the ‘Test’ stimulus: up or down (see FIG. 1).

Paradigm 1.—Motion Discrimination—This paradigm served to measure the instantaneous influence of tACS on coarse motion discrimination. Eight subjects participated in the experiment. The subjects were presented the ‘Long Test’ stimuli and indicated the perceived global direction of motion (up or down). The coherence of the ‘Long Test’ stimuli ranged from −100% (all dots moving down) to +100% (all dots moving up). Stimulation was applied over the left hMT+only during the presentation of the ‘Long Test’ stimuli. In separate sessions, the visual stimulus was either presented in the right hemifield (contralateral condition) or left hemifield (ipsilateral condition).

Paradigm 2.—Motion Adaptation—This paradigm measured the influence of tACS on the induction of adaptation using a standard top-up design. Each experimental session started with a single, 40 sec. presentation of the ‘Long Adapter’ stimulus. In all subsequent trials, the ‘Top-up Adapter’ stimulus (4 s) was followed by a blank period (500 ms) and then by the ‘Test’ stimulus (1 s). The subject's task was to indicate the coherent motion direction of the ‘Test’ stimulus.

In the stimulation conditions, tACS was applied only when the ‘Long Adapter’ or ‘Top-up Adapter’ stimulus was on the screen. In the no-tACS conditions, no stimulation was applied. For the contralateral and ipsilateral experiments, the left hemisphere was stimulated while showing the stimulus on the right hemifield and left hemifield, respectively.

To monitor and control the allocation of attention, subjects were instructed to attend to the adapter stimulus and press a key when a brief (200 ms) doubling of speed occurred (at an unpredictable time). As a secondary benefit, this attention to the adapter also increases the strength of adaptation. Trials in which the subjects failed to detect the speed changes were removed from the analysis.

Paradigm 3.—Recovery—This paradigm probed the influence of tACS on recovery from adaptation. In this experiment, the time between adapter and test (during which the screen was blank) was 4 sec.; in most subjects this still produces a residual aftereffect. In separate sessions, either no tACS was ever applied, or tACS was applied during each 4 sec. blank period.

Paradigm 4. Pre stimulus tACS—This paradigm investigated whether behavioral effects of tACS require the neural changes induced by adaptation. Each trial started with a 4 sec. blank period, followed by an interval of 500 ms and then by the Test stimulus. In separate sessions, stimulation was either always off or on during every 4 sec pre-stimulus blank period.

Data Analysis—Curve fitting—Probit Analysis was used to evaluate the data. The behavioral choice data (proportion of upward choice) was fit with cumulative Gaussians using MATLAB (MathWorks, Natick, Mass.). Binomial noise was assumed on the proportion of up/down responses. The fitted curves all had R2 values above 0.7. The curve fits provided two dependent measures; the point of subjective equality (PSE) and the sensitivity. The PSE was defined as the coherence level at which the fitted curve reached 0.5 and the sensitivity as the slope of the fitted curve at the PSE. The motion after effect (MAE) was qualified as the difference between the PSE of the adapted and unadapted conditions (both in the absence of tACS).

Statistical Analysis—At the single subject level, the inventors used non-parametric permutation tests to determine whether PSEs and sensitivities were significantly different between two conditions (e.g. adapted without tACS and adapted with tACS). In this procedure, the inventors combined the responses from all trials in both conditions, drew (with replacement) two complete datasets from this distribution, and determined the difference in the PSE or sensitivity. The resampling process was repeated 1000 times to obtain a null distribution of the differences. The p-value was then determined of the test as the fraction of values in the null distribution that were larger than the actual difference between the two conditions. Unlike the methods that are derived from asymptotic theory, the bootstrap method is ideal for analyzing psychophysical data because its accuracy does not depend on large numbers of trials, or assumptions (such as normality) about the underlying distributions.

At the group level, a paired Wilcoxon signed rank test was performed separately for the motion discrimination, motion adaptation, recovery from adaptation, and pre-stimulus tACS experiments. For the motion adaptation and the motion discrimination experiments, a two-sided Wilcoxon ranksum test was also used to compare the differences in the changes (sensitivity and PSE) induced by tACS during the contralateral versus the ipsilateral condition. All statistical conclusions remained the same even after the exclusion of the data collected from the non-naïve subject.

Analysis Of The Relation Between Adaptation Strength And tACS-Induced Effects—To investigate whether the influence of tACS (on the PSE or the slope) increased with the strength of adaptation, the inventors calculated the Pearson correlation coefficient (ρ) between the tACS-induced change and the MAE. Specifically, for the change in PSE:

ρ=corr(PSEadapt,tACS-PSEadapt, PSEadapt-PSEunadapt).

A permutation test was used to test the null hypothesis that this correlation was larger for contralateral than for ipsilateral tACS stimulation. A null distribution of differences in correlation was created by randomly sampling PSEs from the ipsilateral and contralateral conditions, and calculating the difference in ρ for 1000 shuffled data sets. A statistically significant difference in correlation between contralateral and ipsilateral tACS was defined as a difference in ρ that was larger than the 95th percentile of this null distribution. The analogous analysis was performed for the sensitivity data.

Results—The influence of tACS (±0.5 mA, 10 Hz) on motion sensitivity and adaptation was measured by applying it at various times during a standard motion discrimination task; during discrimination, before discrimination, during adaptation, and during recovery from adaptation.

tACS Improved Motion Sensitivity—The inventors first tested the hypothesis that tACS injects nuisance perturbations in the motion direction discrimination system. This hypothesis predicts a decrease in the subjects' sensitivity when tACS is applied over hMT+during a motion discrimination task. (See Methods; Paradigm 1). FIG. 2, Section A (bottom) shows the performance of one of the subjects with (thick black curve) and without (thin gray curve) stimulation. The two measures of interest; the Point of Subjective Equality (PSE) and the sensitivity (see Methods), were extracted from the curves. Contrary to the inventors' expectation, transcranial stimulation improved discrimination sensitivity (FIG. 2, Section B, p<0.05, Wilcoxon signed rank test; Cohen's d=0.79; effect size (r)=0.36).

The functioning of area hMT+ is lateralized, that is, the right hemisphere responds primarily to stimuli presented in the left visual field and vice versa. Control experiments were performed to assess the selectivity of tACS and exclude a number of potential confounds. In these experiments, the parietal electrode was placed ipsilateral to the visual stimulus. Assuming that the tACS-induced fields are at least coarsely localized (i.e. within a hemisphere), this should not affect motion processing, hence these experiments control for general changes in arousal or attention induced by tACS (see Discussion hereinafter).

Stimulating the ipsilateral hemisphere did not induce any consistent change in performance (FIG. 2, Sections C-D, p>0.05). Moreover, the sensitivity during contralateral stimulation was significantly larger than during ipsilateral stimulation (two sided Wilcoxon ranksum test, p<0.05; Cohen's d=1.76; effect size (r)=0.66).

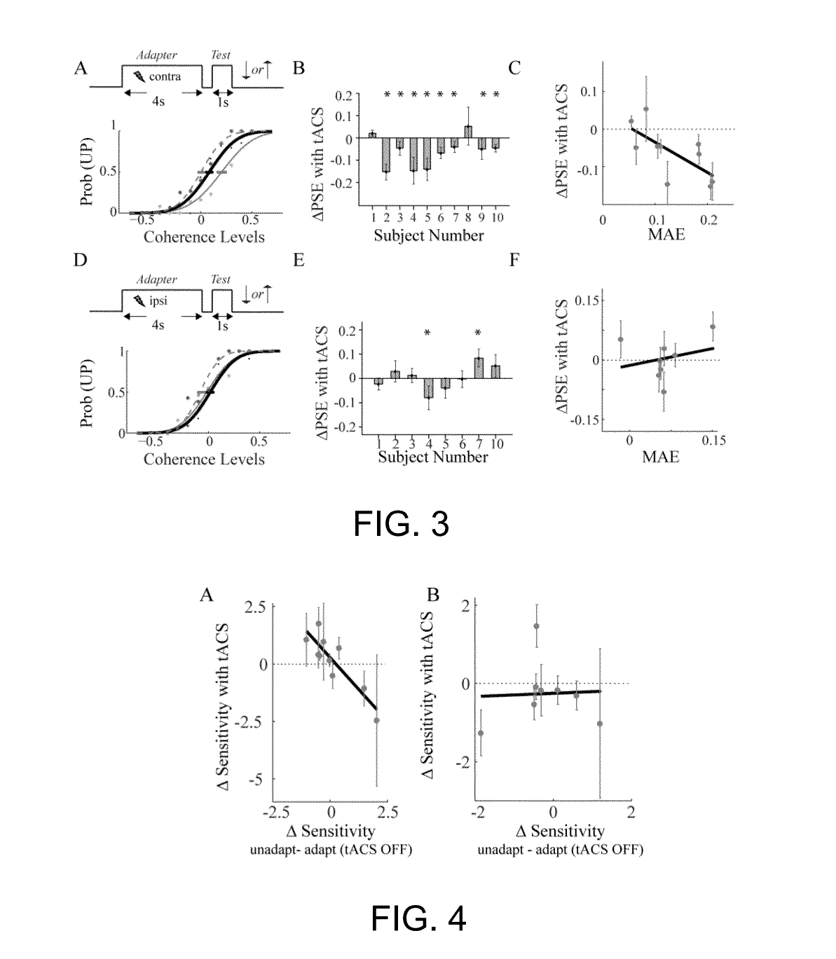

tACS Attenuated The Motion After Effect—In a second set of experiments, the inventors tested the hypothesis that tACS affected a form of plasticity that is reflected in the behavioral changes occurring after prolonged exposure to a moving stimulus. Specifically, the inventors determined psychometric curves for motion discrimination before and after motion adaptation, with and without contralateral or ipsilateral tACS during the adaptation phase (See Methods; Paradigm 2). FIG. 3, Section A (bottom) shows the results for one subject: the dashed curve is the psychometric curve in the unadapted condition. The PSE was at −0.08, which means that this subject reported upward and downward motion equally often when the fraction of downward moving dots was 8% (indicating an upward bias). After adaptation, the (thin solid) psychometric curve was shifted rightward to a PSE of +0.13. Hence, after adaptation, a pattern in which 13% of the dots moved upward was reported to move upward or downward equally often. This is the MAE, which was quantified as the difference in the PSE between the adapted and unadapted condition. For this subject, the MAE size was PSEadapt−PSEunadapt=13%−(−8%)=21%. The thick solid psychometric curve shows the results when tACS was applied during the adaptation phase, this curve is shifted less compared to the unadapted curve, which shows that tACS reduced the MAE. The tACS effect was quantified as the difference in PSE between the stimulated and not-stimulated adaptation condition: PSEadapt,tACS−PSEadapt=−2%−13%=−15%.

Across the group of subjects, the contralateral application of tACS during motion adaptation significantly reduced the MAE (FIG. 3, Sections A-B; p<0.05, Wilcoxon signed rank test; Cohen's d=0.93, effect size(r)=0.42). By comparison, ipsilateral stimulation did not yield a significant change in MAE (FIG. 3, Sections D-E; p>0.05), and a direct comparison showed that the effect of contralateral tACS was significantly larger than ipsilateral tACS (p<0.05; Cohen's d=0.90, effect size (r)=0.41).

Subjects with a large MAE in the absence of tACS typically had a larger reduction in MAE when tACS was applied (FIG. 3, Section C). This negative correlation supports the idea that tACS interferes with the mechanisms of adaptation (FIG. 3, Section C; Pearson correlation coefficient=−0.63). Such a correlation was not found for ipsilateral stimulation (FIG. 3, Section F), and a permutation test confirmed that the correlation induced by contralateral stimulation was significantly larger than that induced by ipsilateral tACS.

tACS Attenuated Sensitivity Changes During Adaptation—Adaptation not only shifted the psychometric curve, it also changed its slope, a measure of subjects' sensitivity to motion. This is consistent with the results of Van Wezel and Britten, who demonstrated that adaptation reduces motion sensitivity. The inventors found a similar reduction in sensitivity (a shallower slope) in most of the subjects. For each of those subjects, tACS increased sensitivity. For two of the subjects, adaptation significantly increased sensitivity; for those subjects tACS decreased sensitivity. This negative correlation is further evidence that tACS attenuates adaptation (FIG. 4, Section A, Pearson correlation is −0.68). This relationship was not found during ipsilateral stimulation (FIG. 4, Section B) and the difference between the contralateral and ipsilateral condition was statistically significant (permutation test; p<0.05; see Methods).

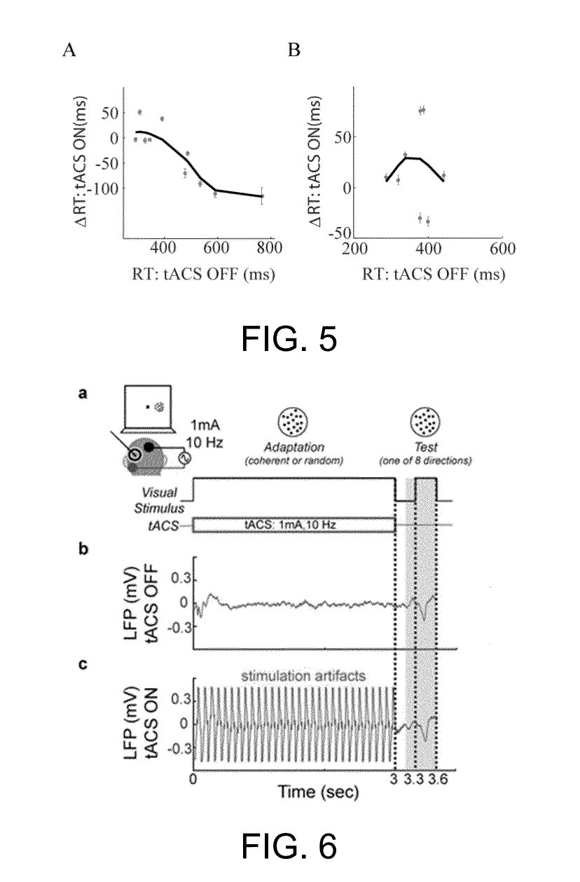

To control and monitor the allocation of attention, the subjects performed a speed detection task during the adaptation phase. This provided an additional and independent measure of motion sensitivity. It was found that contralateral stimulation reduced subjects' reaction time on this task (FIG. 5, Section A). This was mainly driven by subjects whose reaction times were long in the absence of tACS. Ipsilateral stimulation, on the other hand, did not affect the reaction time systematically (FIG. 5, Section B).

tACS Did Not Affect Recovery From Adaptation—In the adaptation paradigm, one can distinguish between an induction phase (the time when the adapter was on the screen) and a recovery phase (defined here as the time between the adapter and the test stimulus, when the screen was blank). The previous experiment showed that tACS during the induction phase reduced the MAE. Here the inventors investigated whether tACS during recovery could also change the MAE.

The duration of the recovery phase (the time between adapter and test) was increased to 4 sec., and tACS was applied only during recovery. In this phase, the subjects had already been adapted to the prior visual stimuli (Top-up Adapter) but they did not receive visual motion input (See Methods; Paradigm 3). Stimulation in the recovery phase had no significant effect on the subsequent MAE nor did it change the slope of the psychometric curves (p>0.05; average ΔPSE=0.01, s.d=0.05), average ΔSensitivity=−0.0067, s.d=0.82). In other words, tACS affected the induction of adaptation (FIG. 3), but not the recovery from adaptation.

tACS Effects Required Motion Adaptation—In the motion adaptation experiments above, tACS was always applied well before the test stimulus (together with the adapter), hence it is possible that simply preceding a test stimulus by tACS induced a behavioral effect and that adaptation was not required per-se. To test this hypothesis, the inventors performed experiments in which each test stimulus was preceded by 4 sec. of a blank screen. tACS was applied only during this blank period (See Methods; paradigm 4). Under these conditions, there was no significant effect of tACS on the PSE or sensitivity (p>0.05; average ΔPSE=0.02, s.d=0.06, average ΔSensitivity=−0.19, s.d=1.33). In other words, when applied outside the adaptation context, tACS had no effect, supporting the interpretation that tACS interfered with adaptation.

Discussion—The inventors investigated how transcranial alternating currents affect human motion perception. The inventors found that tACS reduced motion adaptation and improved motion discrimination sensitivity. Electrical stimulation did not affect motion perception when applied before visual stimulus presentation, or during the recovery phase of adaptation. Taken together, these findings can be summarized succinctly as demonstrating that tACS attenuates the induction of adaptation.

The inventors first address some of the confounding factors and limitations in the interpretation of the data. Then the inventors speculate on the neural mechanisms that could be involved in this and conclude with a brief discussion of the implications of the findings for the practical usage of tACS.

Confounds—Transcranial AC stimulation at 10 Hz can generate phosphenes due to current spread to the retina. As an additional “visual” stimulus that is only present in the tACS conditions, these retinal phosphenes could in principle interfere with adaptation. Several arguments, however, speak against this. First, phosphenes occur in the periphery and—given the receptive field locations of neurons in motion areas—the visual stimulation induced by tACS phosphenes and the motion stimulus affect non-overlapping populations of neurons. Second, tACS induces phosphenes in both hemifields, with no obvious patterns of lateralization. Hence if tACS reduced adaptation by drawing attention away from the adapter, one would expect to find it in both ipsilateral and contralateral stimulation conditions. The control experiment (FIG. 3, Section D), however, shows that only contralateral stimulation reduced adaptation. The specificity of the effect for contralateral tACS also argues that the action of tACS is significantly more pronounced in the cortical hemisphere over which it is applied and is incompatible with a general change in arousal induced directly or indirectly via the generation of phosphenes.

While these experiments used 0.5 mA tACS with a temporal frequency of 10 Hz, follow-up experiments have shown that the enhancement of visual motion detection was ineffective at stimulation frequencies below 5 Hz, but was effective for higher frequencies, at least up to 80 Hz.

Under the particular experimental conditions, the inventors found that tACS increased motion sensitivity. This is incompatible with the view that tACS injects neural noise or perturbations. Of course, one cannot extrapolate such a finding to higher currents, other temporal frequencies, or other stimulation patterns. In fact, it is inevitably the case that at high enough currents, tACS would impact behavioral performance negatively and therefore be behaviorally equivalent to the injection of “noise”.

Comparison with tDCS—Antal et al. have shown that transcranial direct current stimulation (tDCS) over hMT+ reduces the subjective duration of the motion after effect. The goal of the Antal et al. study, however, was not to investigate which aspects of motion adaptation tDCS interferes with, but to provide support for the causal involvement of hMT+ in the MAE. Presumably for this reason, tDCS was applied continuously both during adaptation induction, recovery, and the subsequent motion detection task. Hence, the reduction in MAE duration could have been the consequence of tDCS' interference with any of these processes; this prevents a direct comparison with the findings. Nevertheless, it is of interest to note that Antal and colleagues found that tDCS reduced the MAE irrespective of whether the anode or the cathode was placed over hMT+. This is compatible with the finding that tACS, which also generates current flow of both polarities, attenuates adaptation. The behavioral data cannot address the question whether the same mechanisms underlie the influence of tACS and tDCS, but for tDCS the inventors can speculate that the underlying mechanism is likely different from the (polarity dependent) modulation of excitability reported in motor cortex.

Mechanism—The experiments show that tACS attenuates motion adaptation. Hence, one would expect that tACS attenuates any of the consequences of adaptation. Together with the finding that motion adaptation reduces performance on a coarse motion detection task this provides a succinct explanation of the behavioral changes the inventors observed. For instance, tACS increased sensitivity and reduced reaction times most for those subjects who showed a large adaptation effect (FIG. 4, Section A and FIG. 5, Section A). Importantly, this also accounts for the tACS-induced increase in sensitivity during the presentation of a single RDK (FIG. 2, Section B). Even though this experiment did not involve a separate adaptation stimulus, the 4 second long RDK likely triggered adaptation. The data supports the view that this adaptation was attenuated by tACS, and this led to an increase in sensitivity.

At the circuit level, prolonged exposure to moving stimuli is known to result in firing rate changes throughout visual cortex. Individual neurons can increase or decrease their firing rate with adaptation and this depends critically on the relationship between the tuning of the neuron and the properties of the adapter and test stimuli. For instance, the speed of the moving stimulus, the direction of motion, as well as its size and duration all affect firing rate changes induced in an adaptation protocol. This shows that the consequences of adaptation depend critically on the circuit in which neurons are embedded and implies that the consequences of tACS for a single neuron will also depend strongly on its connections within the local circuit. In other words, based on the behavioral observations and the known properties of adaptation at the single neuron level, it seems unlikely that tACS would generally increase or decrease firing in a population of neurons. The inventors tested this using extracellular recording in the middle temporal area of the macaque during transcranial stimulation (See Third Experiments below) Linking behavioral data with cellular mechanisms requires many assumptions, and is inevitably speculative. Nevertheless, the inventors believe it is valuable to put forward a novel and testable hypothesis that aims to do so. The inventors start from the observation that small membrane voltage fluctuations reduce spike frequency adaptation, as shown by in-vitro recordings of rat hippocampal CA1 neurons using direct somatic current injection. The inventors speculate that tACS could induce such membrane fluctuations in the soma or dendrites and thereby interfere with adaptation.

Third Experiments—Macaque Monkeys

Two adult male rhesus monkeys (Macaca mulatta) participated in these experiments. Experimental and surgical protocols were approved by the Rutgers University Animal Care and Use Committee and complied with guidelines for the humane care and use of laboratory animals of the National Institutes of Health.

Surgical procedures and electrode location—All surgical procedures were conducted under sterile conditions using isoflurane anesthesia. Titanium head posts (Gray Matter Research) were attached to the skull using titanium bone screws. Custom made high-density polyethylene recording chambers were implanted normally to the skull, and dorsal to the expected location of MT. The inventors confirmed recording locations in area MT on the basis of structural magnetic resonance images obtained after implantation, as well as on the basis of physiological criteria such as the high prevalence of direction selective responses, and the relatively small receptive fields (compared to neighboring area MSTd).

Recording—Visual stimulus generation, the triggering of tACS, and data acquisition were under the control of the in house software for visual experimentation: Neurostim (http://neurostim.sourceforge.net). Stimuli were presented on a CRT monitor (Sony GDM-520) spanning 30°×40° at a resolution of 1024×768 pixels and a refresh rate of 150 Hz.

At the beginning of each recording session, the inventors punctured the dura with a sharp, metal guide tube to allow access to the cortex. The guide tube or one of the head screws served as the ground for the electrode signal. The inventors used a micro-positioner (NAN Instruments, Nazareth, Israel) to lower a parylene coated tungsten electrode (1.5 MSΩ; FHC Inc., Bowdoin, Me.) into area MT through the guide tube. The inventors manually isolated single cells by listening to their visually driven response which was made audible on a speaker while the monkey observed moving stimuli (see section “Experimental procedures”). The raw signal was sampled at 25 kHz using Alpha Lab (Alpha-Omega Engineering, Nazareth, Israel). To extract spikes the inventors first band-pass filtered the raw signal between 300 Hz and 6 KHz, and then applied a threshold equal to 4 standard deviations of the filtered signal. The inventors used KlustaKwik to cluster these waveforms into separate units (up to three, significantly direction tuned units per recording depth). Local field potentials (LFPs) were extracted from the raw signal by band-pass filtering between 1 and 120 Hz and then resampling at 781.25 Hz. Eye movements were recorded using an infrared eye tracker (Eyelink2000; SR Research). Trials in which eye position deviated from the fixation point by more than 1° were not used in the analysis.

Transcranial stimulation—Matching the procedures of the second experiments, the inventors delivered tACS with an STG4002 stimulus generator (Multi Channel Systems, Reutlingen, Germany) through 3.2 cm×3.2 cm reusable surface electrodes (uni-tab). The applied current was always sinusoidal with a 1.0 mA amplitude and 10 Hz frequency.

One tACS electrode was placed between the ear and the recording chamber, adjacent to area MT (in the left hemisphere for monkey N and in the right hemisphere for monkey M). The other electrode was placed 4 cm anterior to the vertex. To improve skin conductivity at the site of the electrodes, the inventors applied a mixture of water, isopropanol and aluminum chlorohydrate to the area of the scalp.

Experimental Procedures—In each experiment, the monkey started a trial by bringing its gaze within an invisible 2°×2° window surrounding a small red dot that was permanently present at the center of the screen. The animals were rewarded with apple juice at the end of the trial, for maintaining fixation throughout each trial.

In each recording session, the inventors ran two preliminary mapping experiments to guide stimulus location and motion direction of the main experiment. First, the inventors determined the preferred direction of the neuron using a sparse full screen pattern of dots that moved along a circular path resulting in a uniform translational velocity. Second, the inventors determined the spatial receptive field using localized motion pulses in the preferred-direction in a matrix of 4×3 patches covering the screen. In subsequent experiments, the stimuli were centered on the patch that elicited the maximum mean response.

In the main experiment, trials consisted of a 3 s adapter stimulus followed by a 300 ms blank period in which only the fixation dot was visible, and a 300 ms test stimulus. Both the adapter and test stimulus consisted of 700 anti-aliased dots (30 cd/m2, effective diameter 1.5 pixels) on a 4 cd/m2 background, moving within a 5 o radius circular aperture.

The main experiment was a 2×2 factorial design to test the hypothesis that tACS attenuates adaptation induced changes. The first factor was the level of motion adaptation, which the inventors manipulated by choosing the adapter stimulus. Each of the dots in the adapter stimulus either moved in a randomly chosen direction, or they all moved in the neuron's preferred direction. The random motion stimulus is known to induce much less adaptation than the coherent motion stimulus, hence for ease of reference the inventors will use the terms adapted and unadapted for the levels of this factor. The second factor was the presence or absence of tACS (10 Hz, 1.0 mA); in tACS-ON conditions, it was only applied during the 3 s that the adapter stimulus was on the screen. In the tACS-OFF trials it was not applied at all.

The dots in the test stimulus moved coherently in one of eight evenly spaced directions spanning the circle. This allowed us to measure a direction tuning curve under each of the experimental conditions. The four conditions of the factorial design were presented in separate blocks with one repeat per test-direction. The blocks were randomly interleaved and repeated at least 10 times.

Data Analysis—Tuning Curves—The primary interest was to determine how tACS affected direction tuned responses. The inventors used the average response (firing rate) of a neuron during the 300 ms test interval to estimate tuning curves, separately for each of the four conditions of interest (coherent-adaption/random-adaptation×tACS-ON/tACS-OFF). Using a resampling based Bayesian method the inventors estimated tuning amplitude (TA), tuning width (TW), baseline (un-tuned) firing rate (BS) and preferred direction (PD) of a circular Gaussian tuning curve. From these measures the inventors also extracted the responsivity: the difference between tuning amplitude and its baseline firing rate (TA-BS).

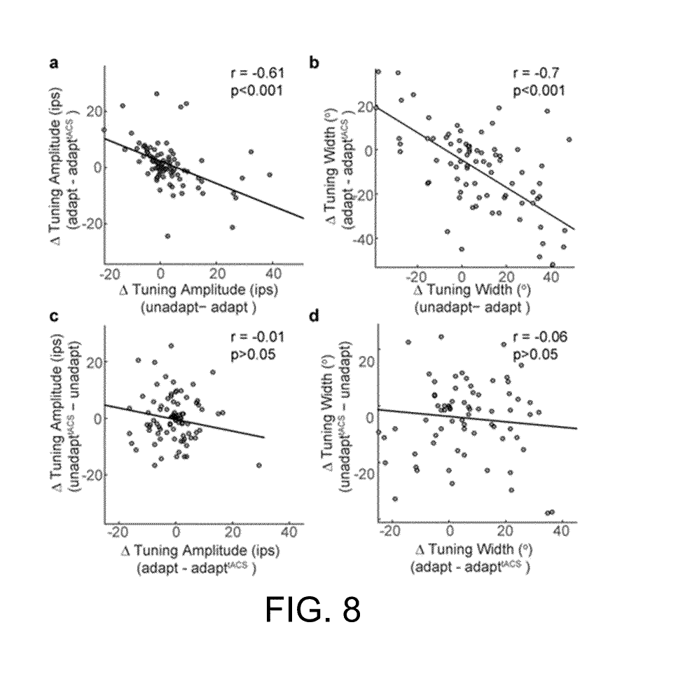

Correlation Analysis—The behavioral results from the second experiments show that the influence of tACS depends on the strength of adaptation. To investigate the neural basis for this effect the inventors determined the Spearman correlation (ρ) between the change in tuning amplitude due to tACS (TAadapt-TAadapttACS) and the change in amplitude due to adaptation (TAunadapt-TAadapt) (refer FIG. 8).

Note that these two measures both depend on coherent motion adaptation without tACS (TAadapt), and are thus not mutually independent. This precludes the use of standard significance testing of Spearman's ρ. Instead, the inventors used a permutation test. The observed data (tuning amplitudes) can be treated as a matrix with N rows and M columns, where N is the number of recorded cells and M is the number of experimental conditions (column 1: unadapted, column 2: adapted and column 3: adapted with tACS). Hence to estimate the significance of ρ=corr(column 1-column 2, column 2-column 3), the inventors computed the null distribution of correlations by randomly shuffling the data matrix 1000 times and estimating ρ for each shuffle. To test for significance, the inventors compared the actual ρ with the 95th percentile of the null distribution. The same analysis method was used for the tuning width data.

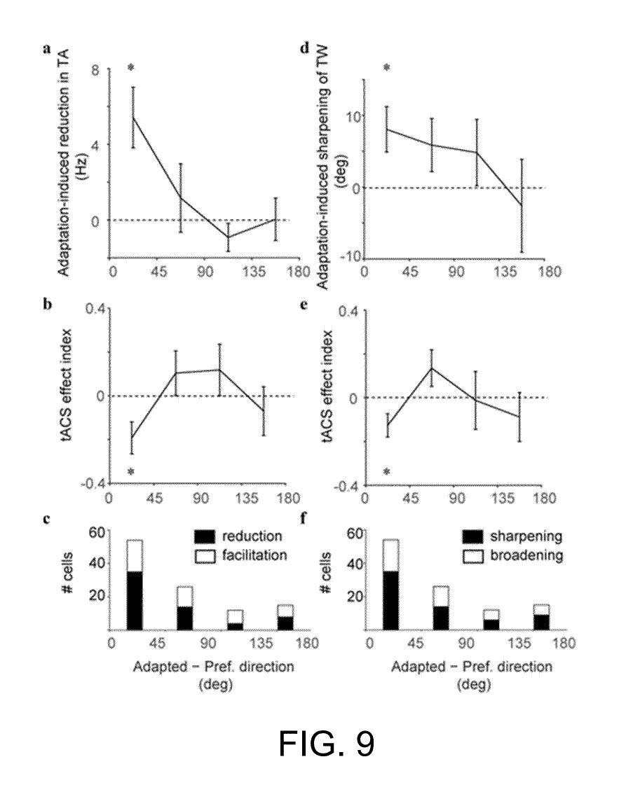

For the data presented in FIG. 9, the neurons were first grouped according to the difference between their preferred direction and the direction of the coherent adapter. Then the above analysis was performed on each group. Each value plotted in Sections b and e of FIG. 9 is the difference between the mean of the null distribution and the actual ρ for each group.

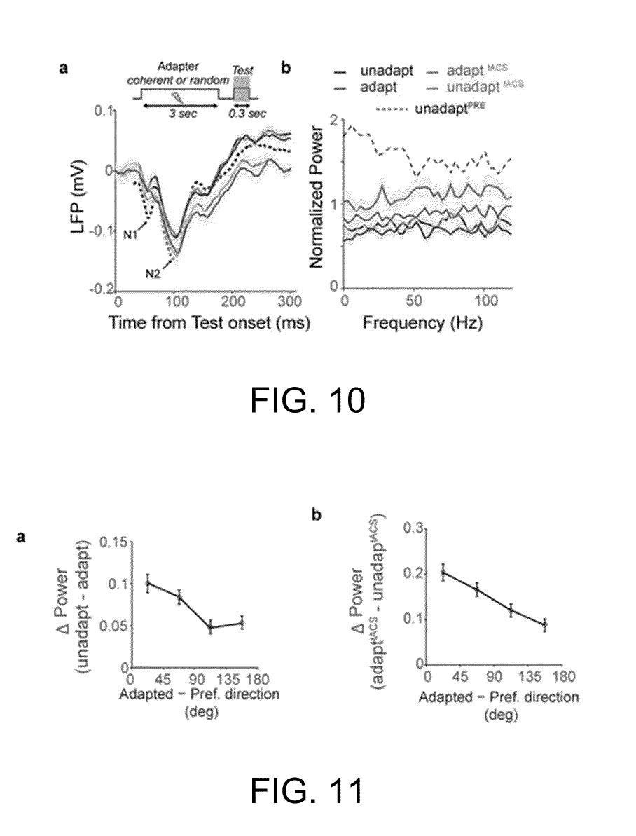

LFP Analysis—Analyses of evoked LFP amplitude—Local field potentials (LFP) were band-pass filtered between 1 and 120 Hz and sampled at 781.25 Hz. The evoked responses were determined by averaging the LFP during test stimulus presentation over all trials corresponding to a given adaptation/stimulation condition. For the average evoked LFP shown in FIG. 10, Section a, the inventors subtracted the response before stimulus onset (i.e. set the response to zero at time 0) and then averaged across all recording sessions. Hence the curves show the net deflection from baseline following stimulus onset. The inventors used a two way ANOVA with factors adaptation (coherent/random) and stimulation (tACS/no tACS) to test if the evoked LFPs were significantly different (in FIG. 10, Section a) across the separate conditions. To quantify the evoked LFPs as a single value, the inventors integrated the absolute value of the raw signal over the Test stimulus duration, for each condition. The inventors observed two negative peaks, N1 (50-70 ms) and N2 (90-110 ms) in the evoked potential. The inventors performed Wilcoxon signed rank test to compare the mean LFP signal in the respective time bands (N1 and N2), across conditions.

Spectral analyses of evoked LFP—For the spectral analysis, the inventors focused on the LFP trace recorded 150 ms post tACS offset. For each recording site, the inventors first calculated the mean evoked LFP for each of the four conditions (coherent-adaption/random-adaptation×tACS-ON/tACS-OFF). The inventors then removed the evoked component by projecting the LFP in each trial onto the mean evoked LFP for the respective condition and keeping only the orthogonal component. Multi-taper spectrograms were estimated using used the Chronux software package with parameters: time bandwidth product=1.2, number of tapers=2, sampling frequency=781.25 Hz. The inventors estimated the power spectrum (frequency range 0-120 Hz) of the mean—evoked potential removed LFP for each trial, across all conditions and sites. For FIG. 10, Section b, the inventors normalized the power spectrum for each site, by dividing the power at each frequency by the mean power at that frequency across all four experimental conditions. To investigate specific frequency bands, the inventors divided the frequencies into five non overlapping bands, alpha (8-15 Hz), beta (15-30 Hz), low gamma (30-50 Hz), medium gamma (50-80 Hz) and high gamma (80-120 Hz). The inventors then calculated the ratio of the power in the tACS conditions with the corresponding no-tACS conditions per frequency band. This approximately equalized the variance across conditions and enabled us to perform a one-way ANOVA with frequency as a factor.

For FIG. 11, the inventors first estimated a tuning curve using the Bayesian method describe above, but now based on the LFP power of the band between 30 and 120 Hz, which has been shown to have directional tuning. The preferred direction of each site was then used to group recordings into 4 bins based on the distance between the direction of the coherent adapter stimulus and the preferred direction of the site.

Results—The inventors recorded extracellularly from 107 motion-selective neurons in two male Macaca mulatta. The tACS electrodes were placed on the scalp over the superior temporal sulcus, one electrode on either side of the implanted recording chamber (FIG. 6, Section a). The monkeys were trained to fixate a dot at the center of the screen and maintain fixation while moving random dot stimuli were presented in the neuron's receptive field.

Based on the previous behavioral findings, the inventors hypothesized that tACS would interfere with the induction of adaptation. The inventors therefore measured direction tuning curves in a strongly adapted state (i.e. after adaptation to 3 s of coherent motion) and in a weakly adapted state (after adaptation to 3 s of random motion; the inventors refer to this condition as unadapted, see Methods). To investigate the influence of tACS on the induction of adaptation, the inventors applied tACS (1 mA, 10 Hz) during the adaptation period in half of the trials (chosen randomly). Stimulation artifacts (FIG. 6, Section c) prevented a meaningful analysis of the extracellular recordings during the adaptation period, and the inventors limited the analysis to the interval between 150 ms after tACS offset and the end of the Test stimulus (FIG. 6, Sections b and c).

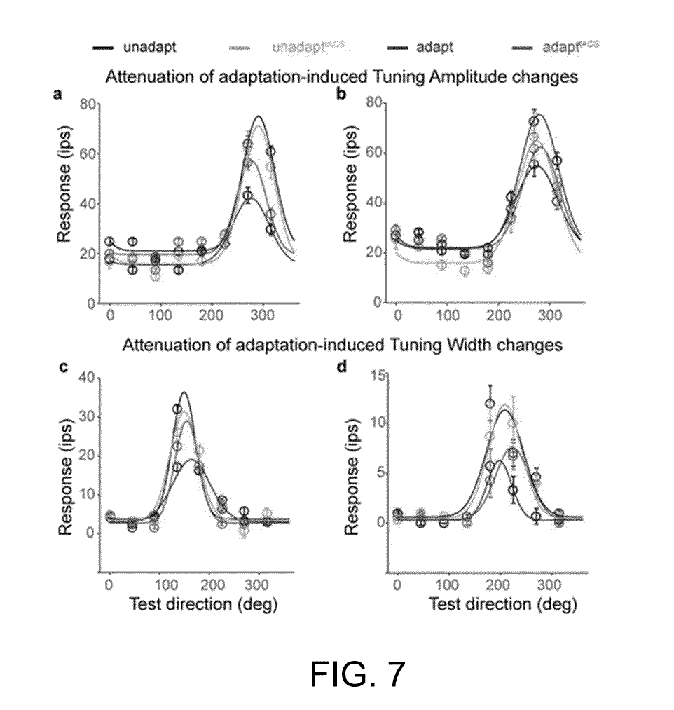

tACS affects tuning curves of single neurons—FIG. 7 shows the tuning curves of four example neurons. The response amplitude of the neuron shown in FIG. 7, Section a, was much reduced following adaptation to coherent motion (adapt, blue curve) compared to following adaptation to random motion (unadapt, black curve). When coherent-motion adaptation was combined with tACS, the amplitude suppression was approximately halved (adapttACS, red curve). FIG. 7, Section b shows an example neuron whose tuning amplitude increased after adaptation. In this neuron, concurrent tACS and adaptation led to a smaller increase in firing rate (FIG. 7, Section b, red curve). Both examples show that tACS attenuated the effects of adaptation on tuning amplitude. The effects of adaptation on the tuning widths were similarly attenuated by tACS. In FIG. 7, Section c, tACS reduced the adaptation-induced broadening of the tuning curve, whereas in FIG. 7, Section d, tACS reduced the adaptation-induced sharpening. When tACS was applied during random-motion adaptation (unadapttACS, green curve) the changes in tuning curve were smaller and not consistent across cells (FIG. 7, Sections a-d).

The effects of tACS are proportional to the level of adaptation—To determine whether the attenuation of adaptation was consistent across the population, the inventors compared the tuning amplitudes and widths of the four tuning curves obtained in each of the 107 neurons in the sample. FIG. 8, Section a shows the relation between the adaptation-induced change in amplitude (horizontal axis in FIG. 8, Section a) and the effect of tACS (vertical axis). There was a significant negative correlation (Spearman correlation, ρ(107)=−0.61; p<0.001), which signifies that tACS attenuated the effect of adaptation regardless of the sign of the effect of adaptation per se. FIG. 8, Section b shows the analogous analysis for changes in the width of the tuning curve. In neurons whose tuning curve was strongly broadened by adaptation, tACS led to a narrowing of the tuning curve and in neurons whose tuning curves became narrower after adaptation, tACS led to a broadening. This correlation was also significantly negative (Spearman correlation, ρ(107)=−0.7; p<0.001), and provides additional support for the hypothesis that tACS attenuates adaptation.

An alternative hypothesis for the tACS-induced changes in tuning curves could be that tACS generated an unspecific change, unrelated to adaptation (e.g., an arousal or attentional signal that varied across neurons). If this were the case, one would expect tuning curve changes to be similar when tACS was applied in the adapted and unadapted conditions. The data do not support this hypothesis. FIG. 8, Sections c and d show that, across the population, the tACS-induced tuning changes in the unadapted conditions were not correlated with the changes in the adapted conditions (Tuning Amplitude: ρ(107)=−0.01; p>0.05, Tuning Width: ρ(107)=−0.06; p>0.05).

If tACS indeed interferes with the induction of adaptation, one would expect the largest effects of tACS to occur when adaptation is strong. FIG. 8 supports this view on a population basis, but the recordings also allow us to investigate this in a somewhat different manner. Adaptation effects are known to be strongest when the adapting stimulus is similar to the preferred stimulus. Because the inventors occasionally recorded more than one neuron simultaneously at the same electrode (and because the online estimate of the preferred direction was relatively coarse; see Methods), some neurons were not adapted at their preferred direction of motion.

To leverage this effect, the inventors divided the neurons in four groups on the basis of the angle between their preferred direction and the direction of the coherent adapter (FIG. 9). As expected, neurons showed the strongest reduction in tuning amplitude after exposure to coherent motion within 45° of their preferred direction (FIG. 9, Section a). In agreement with the attenuation hypothesis, the effect of tACS (see Methods) was largest for these neurons (FIG. 9, Section b). Neurons exposed to an adapter further away from their preferred direction adapted less, and tACS had less of an effect. The analogous relationship for changes in tuning width is shown in FIG. 9, Sections d-f. This analysis confirms the hypothesis that tACS effects depend on the level of adaptation.

tACS modulates evoked local field potentials—The inventors used the local field potentials to gain insight into tACS-induced changes in aggregate population activity. FIG. 10, Section a shows the evoked LFPs averaged over recording sites (see Methods). The evoked responses to the adapted and unadapted test stimulus (solid black and blue curve) were similar. The inventors attribute this to the fact that many neurons, with a potentially large range of preferred directions, contribute to these evoked responses. Hence, a random motion stimulus that weakly adapted many neurons could result in the same change as a coherent motion stimulus that strongly adapted a subset of neurons. In addition, adaptation effects that are not specific to the direction of motion (e.g. contrast adaptation) would sum in the large population contributing to the LFP and dominate the direction-specific effects seen more clearly in the single neuron responses. In other words, the terms adapted and unadapted, although commonly used for the analysis of single units, are somewhat of a misnomer for the LFP analysis; both responses to the test stimulus are in fact ‘adapted’.

The inventors confirmed this by analyzing the response to the adapter stimulus. Because the adapter was always presented at the start of a trial, it appeared after a period of very low visual stimulation; the inter-trial interval during which the animal would also often blink or move its eyes. In other words, this response to the first stimulus in a trial provided a better estimate of a truly unadapted evoked LFP response (unadaptPRE). Comparing this unadapted evoked LFP response (dashed black curve) with the test-evoked responses (solid black and blue curve) shows that adaptation led to a reduced evoked response. Consistent with findings in visually evoked potentials in humans, adaptation led to a statistically significant (p<0.05, Wilcoxon signed rank test) reduction in the first and second negativity N1 (50-70 ms) and N2 (90-110 ms).

The test-evoked responses in the tACS trials (red and green curves) show three distinct effects. First, the test-evoked N2 component in the tACS trials was as strong as the adapter-evoked N2 (dashed curves). In other words, tACS attenuated the effect of adaptation on the N2 component. Second, tACS increased the later part of the evoked potential (>100 ms after stimulus onset). In this phase, adaptation had little effect, i.e., the blue, black, and dashed black curves overlap; hence this particular effect demonstrates that at least some tACS-induced neural changes do not require strong adaptation.

tACS increases broadband spectral power—One of the potential advantages of tACS over transcranial direct current stimulation (tDCS) is that it may be able to entrain cortical rhythms at the frequency of stimulation beyond the period of stimulation. The inventors investigated this claim using spectral analysis of the LFP. To avoid contamination by stimulation artifacts, the inventors only considered LFPs recorded at least 150 ms after the offset of stimulation.

To analyze the spectral content during stimulus presentation, the inventors regressed out the average evoked response (i.e. the data leading to FIG. 10, Section a) from each trial and then estimated the power per frequency band (see Methods). As is apparent in FIG. 10, Section b, the LFP power of the response to the adapter stimulus (black dashed curve) was much higher than the power of the response to the test stimuli (blue and black curves) across the entire spectrum. As in the evoked potential, this reduction was similar regardless of whether the adapter was a coherent motion stimulus (blue curve) or a random motion stimulus (black curve). The red and green curves in FIG. 10, Section b show that the spectral power over a broad range of frequencies was larger if tACS was applied during the adaptation phase. This broadband increase in power did not depend on the presence of a visual stimulus as it was also observed in the spontaneous LFP recorded in the 150 ms before the onset of the test stimulus (not shown).

To further investigate the dependence of this power increase on adaptation, the inventors separated recording sites on the basis of their preferred direction (the circular mean of the gamma tuning-curve, i.e., the power measured in the gamma band (30-120 Hz) as a function of motion direction; see Methods). FIG. 11, Section a shows the broadband LFP power was larger in the unadapted compared to the adapted conditions (Δ Power >0). Similar to the spike rate analysis (FIG. 9), sites that were adapted near their preferred direction adapted most. FIG. 11, Section b shows the influence of tACS on the LFP power. tACS increased LFP power most in those sites in which adaptation reduced the power most.

Statistically, the inventors compared the power in the LFP in the test phase using a three-way ANOVA with factors of adaptation (coherent/random) and stimulation (tACS/no-tACS) and frequency. The main effect of tACS was significant both during and before test stimulus presentation. (F(2,1)>100; p<0.001). During test stimulus presentation, both the main effect of adaptation (F(2,1)=11.96; p<0.001) and the interaction between adaptation and tACS (F(2,1)=106.6; p<0.001) were significant. There was no main or interaction effect of LFP frequency (F(2,39)<0.001; p>0.05). This demonstrates that tACS induced an adaptation-dependent broad-band increase in spectral power both in the spontaneous activity and the ongoing activity during stimulus presentation that outlasted tACS offset by at least 300 ms.

tACS did not evoke long-lasting frequency-specific entrainment—Given that the tACS frequency was 10 Hz one might expect entrainment specifically in the alpha band (8-12 Hz). The data do not support this prediction. First, the power spectra in FIG. 5b show little evidence of such frequency-specific entrainment (i.e., no peaks near 10 Hz). Second, the inventors tested this hypothesis quantitatively. The inventors divided the frequencies into five non overlapping bands, alpha (8-15 Hz), beta (15-30 Hz), low gamma (30-50 Hz), medium gamma (50-80 Hz) and high gamma (80-120 Hz). The inventors calculated the ratio of the power in the tACS conditions with the corresponding no-tACS conditions per frequency band. This approximately equalized the variance across conditions and enabled us to perform a one-way ANOVA with frequency as a factor. The main effect of frequency was not significant either before or during test stimulus presentation (F(4)<0.001; p>0.05). This shows that the increase in spectral power was indeed broad-band and that there was no evidence of long-lasting, frequency-specific entrainment of neural activity after tACS offset.

Discussion

The data reveal, for the first time, a number of ways in which transcranial alternating current stimulation affects neural activity in the brain of an awake, behaving primate. Several findings support the hypothesis that tACS interferes with the mechanisms of adaptation. First, the inventors found that tACS attenuated the effects of visual motion adaptation on the peak and width of the tuning curves of single neurons. The impact of tACS was strongest on neurons that adapted most. Second, the inventors found that tACS attenuated the adaptation-induced reduction of the N2 component of the evoked local field potential. Third, the inventors found broad-band increases in spectral power that persisted for at least 300 ms after tACS-offset. This effect was most pronounced at sites where adaptation was strongest.

Phosphenes—In nearly every electrode montage, tACS at 10 Hz and 1 mA evokes phosphenes in most humans and these phosphenes have a retinal origin. This is a potentially problematic confound in tACS studies as the phosphenes generate an additional visual input which may interact with the experimental stimuli directly or influence the subject's attentional state. We have previously argued, based primarily on the peripheral location of the phosphenes and the hemispheric lateralization of the behavioral effects, that the attenuation of adaptation is unlikely to be caused by phosphenes. The current electrophysiological findings add additional arguments against the hypothesis that a general change in attention is responsible for the behavioral and neural consequences of tACS. If the effects of tACS were mediated through attention, the effects should have been observed in the unadapted conditions as well as the adapted conditions. However, the inventors found that the effects of tACS depended critically on the presence of adaptation.

Long-Term Entrainment—Several recent animal studies have shown that neural activity can be entrained to the tACS frequency during the application of tACS. Consistent with the findings, those experiments also reported a lack of long-lasting entrainment after tACS offset. However, Helfrich et al. recently reported that 10-Hz tACS entrained human EEG for up to about one minute after tACS offset. There are a number of possible explanations for this discrepancy. First, the sources of the alpha rhythm in the EEG and the LFP might be different. Second, it is possible that the longer period of continuous stimulation (20 min) in the Helfrich et al. study induced stronger entrainment. Third, the human subjects had a strong ongoing alpha rhythm even before stimulation whereas such a peak in the response was not present in the recordings (not shown). It may be easier for tACS to boost an already present rhythm than establish one de-novo. The fourth possibility, however, is a potentially confounding factor of time in the human EEG experiments: post-tACS EEG measurement followed post-sham EEG measurement by ˜40 minutes. As a consequence, increased drowsiness may have led to an increase in alpha band power. The increase in alpha observed during sham stimulation of the human subjects provides some experimental support for this interpretation. In the study, tACS and no-tACS blocks were randomly interleaved, which would eliminate this confound if it exists.

Attenuating Adaptation—Adaptation is traditionally associated with a reduction in firing rate following repetitive sensory stimulation. But, more recent work in V1, MT and the barrel cortex has shown that responses can also increase after adaptation. The data provide further experimental support for this view. On average, the sample of neurons had a lower peak firing rate (tuning amplitude) after adaptation, but many neurons individually increased their firing rates (See FIG. 7, Section b). The mechanisms underlying this diversity are not fully understood, but it seems likely that suppression of the surround and subsequent disinhibition and the complex consequences of reducing the firing rate of a subset of neurons in the recurrently connected network of motion detectors are involved. This complexity has potential consequences for the fMRI adaptation method which typically assumes that firing rate can only go down after adaptation, as well as for the general understanding of how the brain reacts to its recent sensory experience. For the present study, the heterogeneity of the sign of the effect of adaptation on firing rates provided a benefit as it showed that the consequences of tACS are better understood as an attenuation of adaptation than an overall increase (or decrease) of neural responses.

The attenuation of adaptation hypothesis also provides some insight into the question how tACS can have specific behavioral effects even though it generates electric field changes throughout the brain. If tACS affects only adaptation, then it will affect only those neurons that are undergoing adaptation at the time of tACS stimulation. This implies that the subset of affected neurons can be changed by changing the experimental task that the subject performs while undergoing tACS. In other words, to target a brain area, the experimenter can choose to adapt it with a relevant stimulus or task, which will make it more susceptible to tACS. This novel insight has obvious practical implications for the clinical usage of tACS.

Cellular mechanisms—At the current levels generally considered safe (<2 mA), transcranial electric stimulation generates intracranial electric fields on the order of 1 V/m, and membrane potential changes on the order of at best a few mV. This small effect has led to some skepticism about the efficacy of transcranial electrical stimulation. The results suggest a possible resolution of this issue, because even small (<2 mV) membrane voltage fluctuations have been shown to reduce spike frequency adaptation. This was demonstrated in rat hippocampal CA1 neurons, but the inventors speculate that in the recordings the small tACS-induced membrane fluctuations may have similarly prevented the activation of sodium or calcium activated potassium channels in visual cortical neurons. This would attenuate spike rate adaptation in those neurons and thereby indirectly cause much larger changes in firing rate than expected from a few millivolts of membrane polarization. This hypothesis could be tested in-vitro.

tACS in non-human primates—The non-human primate model provides a unique opportunity to study how transcranial stimulation affects neural processing. Given the gross physical and anatomical similarities, current spread in the macaque is expected to be more similar to the human brain than, for instance, in the lissencephalic rodent brain.

The surgical implants needed to perform intracranial recordings in any animal inevitably modify the induced fields and this could in principle change the effect of tACS. However, both computational models and behavioral studies, predict broad intracranial current spread, hence at least some induced fields reach most of the cortex regardless of the physical details of the implants. The finding that tACS changes neural responses in area MT support this view. Moreover, given that the electrophysiological results were at least broadly consistent with the earlier behavioral findings, the differences between the electric fields the inventors induced in the macaque and human must have been relatively insignificant in terms of the overall effects they evoked.

Implications for tACS usage—Current understanding of direct current stimulation (tDCS) is that the orientation of a neuron in the applied field determines whether its excitability will be increased or decreased through a net depolarization or hyperpolarization of the soma. If, however, tACS influences neurons through the interaction of sub threshold oscillations with adaptation, then it would be affected less by the orientation of the neuron in the field. Given that field orientation in a target area is highly idiosyncratic and difficult to predict, this could be a considerable practical advantage of tACS over tDCS.

In summary, the inventors have discovered that the application of a low levels of alternating current (<2 mA) in the range between 5 and 80 Hz between two scalp electrodes, one placed above visual cortex (for example, T5 in the 10-20 system), the other on the vertex, enhances the visual detection of motion. Application of the transcranial current allowed subjects to perform much better, with improvements in performance of 10% to 30%. The experiments demonstrate an improvement of motion detection but the inventors expect that this same technique could also enhance visual detection of form and the detection of other faint sensory stimuli (sounds, smells).

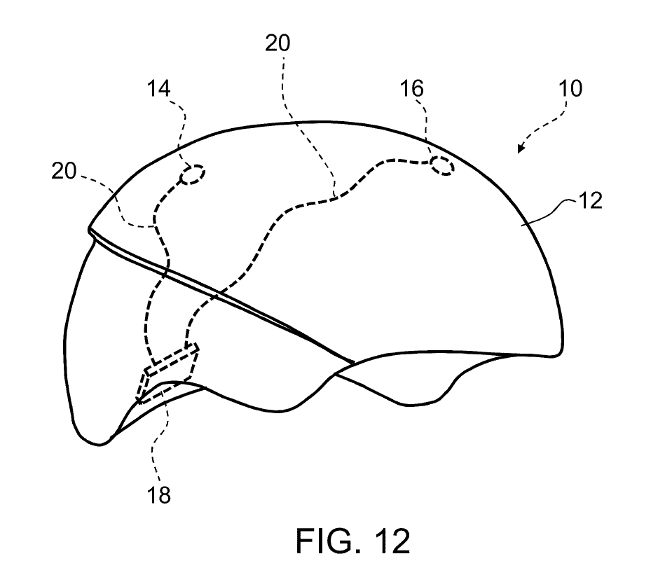

Commercial use of this invention would use headgear or like device to position the electrodes over visual areas. Referring to FIG. 12, an illustrative headgear 10 in accordance with an embodiment of the invention will be described. The headgear 10 has a support shell 12. In the illustrated embodiment, the shell 12 has the form of a helmet, but the invention is not limited to such and the shell may have other configurations, for example, a cap, a stocking or the like. The shell 12 supports a first electrode 14 configured to be placed above visual cortex and a second electrode 16 configured to be placed on the vertex. A power source 18 is also provided and is configured to apply low levels of alternating current (<2 mA) in the range between 5 and 80 Hz between the two electrodes 14, 16, for example, via wires 20, however, the system may alternatively be wireless.

One target for such a headgear device is the computer gamer, who is keen to enhance their visual detection. It may also be possible to develop this technique for other applications that require high visual detection performance such as the monitoring of flight patterns, or the visual detection of anomalies in radiology. Furthermore, the technique may be utilized for enhancement of other sensory modalities. For instance, tACS could help to improve the detection of any faint visual stimulus, for example, a faint sound, touch or smell.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. A system for enhancing a sensory modality of a subject, comprising:
a first scalp electrode configured to be placed above the sensory cortex of the subject;
a second electrode configured to be placed on the subject; and
a power source configured to apply low levels of alternating current above 5 Hz between the first and second electrodes.

2. The system according to claim 1, wherein the low levels of alternating current are less than 2 mA.

3. The system according to claim 1, wherein the low levels of alternating current are applied between 5 to 80 Hz.

4. The system according to claim 1, wherein the sensory modality is visual detection.

5. The system according to claim 1, wherein the visual detection relates to motion.

6. The system according to claim 1, wherein the sensory modality is sound detection.

7. The system according to claim 1, wherein the sensory modality is touch detection.

8. The system according to clam 1, wherein the sensory modality is taste detection.

9. The system according to claim 1, wherein the first and second electrodes are positioned on a shell of a headgear device.

10. The system according to claim 9, wherein the shell defines a helmet.

11. The system according to claim 9, wherein the shell also supports the power source.

12. The system according to claim 11, wherein wires extend between the power source and the first and second electrodes.

13. A method of enhancing a sensory modality of a subject, the method comprising the steps of:
positioning a first scalp electrode above the sensory cortex of the subject;
positioning a second electrode placed on the subject; and
applying low levels of alternating current above 5 Hz between the first and second electrodes.

14. The method according to claim 13, wherein the low levels of alternating current are less than 2 mA.

15. The method according to claim 13, wherein the low levels of alternating current are applied between 5 to 80 Hz.

16. The method according to claim 13, wherein the sensory modality is one of visual detection, sound detection, touch detection or taste detection.

17. The method according to claim 13, wherein the first and second electrodes are positioned on a shell of a headgear device and the steps of positioning the first and second electrodes includes positioning the headgear on the subject.
